(19) United States
(12) Patent Application Publication
(10) Pub. No.: US 2026/0168948 A1
(43) Pub. Date: Jun. 18, 2026

(54) SENSOR UNIT FOR USE IN AN AIR FILTER SYSTEM AND METHOD OF MANUFACTURING THE SENSOR UNIT

(71) Applicants: MANN+HUMMEL GmbH, Ludwigsburg (DE); Institut Fuer Umwelt & Energie, Technik & Analytik E.V., Duisburg (DE); fem Forschungsinstitut, Schwäbisch Gmünd (DE)

(72) Inventors: Michael HARENBROCK, Ludwigsburg (DE); Holger SRADNICK, Ditzingen (DE); Tanja HEILMANN, Meckenheim (DE); Ahmed BANKODAD, Remscheid (DE); Stefan HAEP, Dinslaken (DE); Siegfried OPIOLKA, Duisburg (DE); Alpesh Laxman VORA, Duisburg (DE); Ann-Kathrin EGETENMEYER, Lauchheim (DE); Gloria LANZINGER, Aalen (DE); Birger FREISINGER, Vellberg (DE)

(21) Appl. No.: 19/451,048

(22) Filed: Jan. 16, 2026

(30) Foreign Application Priority Data
Dec. 18, 2024 (EP) 24220872
Dec. 9, 2025 (EP) 25221706

(51) Int. Cl. G01N 27/12; B01D 53/04; G01N 33/00

(52) U.S. Cl. CPC G01N 27/125; B01D 53/0454; G01N 33/0027; B01D 2257/302; B01D 2257/404; B01D 2257/406; B01D 2258/06

(57) ABSTRACT

A sensor unit for use in an air filter system operable to separate at least one harmful gas from an air flow. The sensor unit includes at least a first sensor. The first sensor includes two electrodes and a material layer which connects the two electrodes. The electrical resistance of the material layer changes when the material layer comes into contact with the at least one harmful gas. The sensor unit further includes a processing unit configured to determine a time derivative of a change in resistance of the material layer of at least the first sensor and to generate an output signal based on a function of the time derivative determined by the processing unit. A method for producing the sensor unit includes subjecting the material layer of the first sensor to a surface treatment with an acid.

### CROSS-REFERENCE TO RELATED APPLICATIONS

This application claims the benefit of European Patent Application No. 24220872.6 filed on Dec. 18, 2024, and European Patent Application No. 25221706.2 filed on Dec. 9, 2025, the entire contents of which are incorporated herein by reference to the fullest extent permissible.

### TECHNICAL FIELD

The present disclosure relates to a sensor unit that may be used in in combination with an air filter. The air filter is used to separate a harmful gas such as NH₃ or NO₂ from an air flow. The present disclosure also relates to a method for manufacturing the sensor unit.

### BACKGROUND

The operational life of a fuel cell depends to a large extent on the cleanliness of the air, which is necessary for the chemical reaction that takes place in the fuel cell to generate electrical energy. Cathode air filters are therefore used to separate harmful gases such as NH₃, NO₂ and SO₂, which reduce the operational life of the fuel cell, from the air flow for the fuel cell. Cathode air filters with filter elements containing activated carbon to adsorb the pollutants are used for this purpose.

As the adsorbent gets loaded, complete adsorption of the target gas is no longer possible, resulting in a breakthrough of the gas through the filter. The harmful gases then enter the fuel cell. This gas breakthrough should be avoided to prevent damage to the fuel cell. The sensor unit is used to detect the gas breakthrough. If the sensor unit detects a gas breakthrough, the contaminant-loaded filter element should be replaced. This enables predictive maintenance of the air filter.

### SUMMARY

The present disclosure is therefore based on the object to provide a sensor unit which may be used in an air filter system, which may be manufactured cost-effectively, and which enables reliable detection of harmful gases, for example, NH₃ and NO₂, thus providing an indication for a filter element change.

This may enable predictive maintenance of the air filter system. The sensor unit may provide a warning when the saturation level of the filter element exceeds a saturation threshold. For example, the saturation threshold may be about 80%. The saturation threshold may be tailored to the application and operation mode of the air filter system.

The application of the sensor unit is not limited to fuel cell systems but may also be used in systems facing similar challenges.

The object underlying the present disclosure is solved by the features disclosed herein. Embodiments of the present disclosure may be taken from the descriptions and the accompanying drawings.

According to the present disclosure, it is provided that the sensor unit has a processing unit. The processing unit is designed to determine a time derivative of a change in an electrical resistance of the material layer of the first sensor. The processing unit then generates an output signal based on a function of the time derivative of the change in resistance of the first sensor. The output signal may be used to send a message to the operator of the air filter or to the operator of the air filter/fuel cell combination, for example, that the amount of harmful gases detected is too high and that the filter element should be replaced. The output signal may take a wide variety of forms. It may be acoustic, visual or in the form of a data packet, which is then transmitted to a device for further processing via a suitable interface.

The material layer of the first sensor may be a polyaniline (pani) or a metal oxide (MOx). The sensor unit may have at least a second sensor, whereby the material layer of the first sensor may be different from the material layer of the second sensor. The different sensors react differently to different harmful gases, making it easier to differentiate between the different harmful gases.

In an embodiment, the time derivative or the speed of the change in resistance for example, in the form of the time derivative of the relative change in resistance at the first sensor and/or second sensor is used. When determining the relative change in resistance, the measured resistance R is related to, for example, an initial resistance R0 of the material layer. The initial resistance R0 may be the resistance that the material layer made of polyaniline or metal oxide has before initial exposure to the harmful gas. If the resistance R increases by 20% compared to the initial resistance R0 as a result of exposure to the harmful gas, for example, the relative change in resistance R/R0 assumes the value 1.2.

In the case of the harmful gas NH₃, it has been found that with a time-limited exposure to the harmful gas (for example with an exposure duration of one minute or several minutes), the value R/R0 rises directly at the beginning of the exposure and falls again after the end of the exposure, but does not return to the initial value (R/R0=1) even after a longer period of time after the exposure. Due to this drift of the sensor signal, which adds up if the time-limited exposure is repeated several times with corresponding pauses, a simple correlation between the amount of harmful gas during the exposure and the sensor response based on the relative change in resistance is not possible. If, on the other hand, the time derivative of the relative change in resistance is used, the drift of the sensor signal does not affect the measurement results. By using the time derivative of the relative change in resistance Δ(R/R0)/Δt, the influence of the sensor signal drift observed with NH₃ may be eliminated and the measurement accuracy improved.

If the concentration of NH₃ is continuously increased from exposure to exposure, a good approximation of a linear correlation between the harmful gas quantity/harmful gas concentration and the time derivative is obtained in one embodiment. An increase in R/R0 may be recognized when the exposure duration is increased. This leads to problems if a correlation between R/R0 and the harmful gas concentration is to be established. The time derivative Δ(R/R0)/Δt, on the other hand, is independent of the exposure duration.

Another advantage of using the time derivative of the change in resistance is that mathematical compensation of the influence of relative humidity and temperature is not necessary. In practice, the time derivative of the relative change in resistance is independent of the influence of relative humidity and temperature, as both environmental parameters only change comparatively slowly in practice. The use of the time derivative of the change in resistance increases the measurement accuracy and simplifies the handling of the sensor unit.

In the case of the harmful gas NO₂, it has been shown that the resistance of the polyaniline material layer also increases as a result of a temporary exposure to the harmful gas, but that the resistance remains practically constant after the end of the exposure, even after a longer waiting period, and does not drop back to R0. When re-exposed to the same concentration of harmful gas, R/R0 increases again by the same order of magnitude, so that a cascaded increase in R/R0 may be recognized. This makes it difficult to find a suitable correlation between R/R0 and the harmful gas concentration. However, if the time derivative Δ(R/R0)/Δt is used, it is easier to draw conclusions about the harmful gas concentration.

In one example, the processing unit is designed to generate the output signal depending on the condition as to whether the change in resistance of the material layer of the first sensor exceeds a threshold value S1. Only if, for example, the relative change in resistance R/R0 is greater than S1 (and other conditions may be fulfilled) does the processing unit of the sensor unit generate the output signal.

The processing unit may be designed to generate the output signal depending on the condition as to whether the time derivative of the change in resistance of the first sensor exceeds a threshold value S2. In one example, the output signal depends only on this condition. However, the generation of the output signal may also depend on further conditions, for example, on the condition that the change in resistance is greater than S1.

The processing unit may be designed to generate the output signal depending on the condition whether the time derivative of the change in resistance of the material layer of the first sensor (and optionally, also of the second sensor) falls below a negative threshold value S3. This criterion may be used to differentiate between NH₃ and NO₂. While the resistance of the material layer of a sensor decreases again after exposure to NH₃ has ended and the time derivative of the relative change in resistance therefore assumes values less than zero, the resistance remains at an increased level during exposure to NO₂ and does not decrease even when the exposure has ended and NO₂ is no longer present on the material layer. Accordingly, the time derivative here shows practically no negative values or only negative values that are close to zero, but whose absolute value is not greater than the absolute value of the negative threshold value S3.

In an embodiment, the output signal includes a dynamic resistance ratio R/R_{ref} or the output signal is based on the dynamic resistance ratio R/R_{ref}, where R is the measured resistance of the material layer and R_{ref} is a dynamic baseline resistance. The dynamic baseline resistance R_{ref} depends on the time derivative of the change in resistance of the material layer, for example, on the time derivative of the relative change in resistance and may change over time. By using the dynamic resistance ratio R/R_{ref} for the output signal or as the output signal, a drift of the sensor signal, which may also be caused by changes in relative humidity or temperature, may be avoided or significantly reduced.

The dynamic baseline resistance R_{ref} is defined as follows in an embodiment: R_{ref} corresponds to the measured and time-varying resistance R or R(t) of the material layer of a sensor, as long as the time derivative of the relative change in resistance Δ(R/R0)/Δt, with R0 as the initial resistance, is less than a start threshold value Z1. If the time derivative of the relative change in resistance Δ(R/R0)/Δt remains below this start threshold value Z1, the ratio of the measured resistance R to the dynamic baseline resistance R_{ref} is equal to 1, since the baseline resistance R_{ref} corresponds to the measured resistance R.

From the point at which the time derivative of the relative change in resistance Δ(R/R0)/Δt exceeds the start threshold value Z1, the dynamic baseline resistance R_{ref} is based on the resistance R(Z1) measured at the point at which the start threshold value Z1 is exceeded. The baseline resistance R_{ref} is now a temporary constant, so that the dynamic resistance ratio R/R_{ref} now deviates from the value 1 when the measured resistance R changes over time. As long as the value does not fall below an end threshold value Z2, the dynamic baseline resistance remains at the value R(Z1) or, more precisely, at the value R(tZ1).

While the start threshold value Z1 is positive, the end threshold value Z2 is negative or less than zero. The positive start threshold value Z1 is exceeded when harmful gas exposure begins and the resistance in the material coating increases relatively quickly as a result. When the harmful gas exposure ends, the resistance of the material layer quickly decreases again, causing the value to fall below the end threshold value Z2.

In an embodiment, the start threshold value Z1 and the end threshold value Z2 are equal in magnitude. In an alternative embodiment, the ratio of the magnitudes of the start threshold value Z1 to the end threshold value Z2 is in a range from 0.5 to 2.

The dynamic baseline resistance R_{ref} corresponds again to the measured resistance R(t) as soon as the value falls below the end threshold value Z2. The ratio of R(t) to R_{ref} is now 1 again and remains at this value until the time derivative of the relative change in resistance change Δ(R/R0)/Δt exceeds the start threshold value Z1 (again). In this case, R_{ref} would then be set to the resistance value measured at the time the start threshold value Z1 is exceeded again. If Δ(R/R0)/Δt then falls below the end threshold value Z2 again later, the measured resistance R(t) is used again as the dynamic baseline resistance R_{ref}, with the result that the ratio R(t)/ R_{ref} is then equal to 1 again.

In an embodiment, the output signal includes a concentration of the harmful gas (e.g. expressed in ppb or ppm), wherein the concentration is based on a calibration using the dynamic resistance R/R_{ref}.

A further object of the present disclosure, the provision of a method for manufacturing the sensor unit described above, is solved by the features disclosed herein. Embodiments may be taken from the descriptions and the accompanying drawings.

The material layer of the first sensor may be subjected to a surface treatment with an acid. Insofar as the two sensors and their material layer have been manufactured from polyaniline in an identical manner, this surface treatment constitutes the difference between the two material layers. In an embodiment, the surface treatment is only carried out on the material layer of the first sensor, while no surface treatment is provided for the material layer of the second sensor. However, it is also conceivable that the material layer of the second sensor also undergoes a surface treatment that is different from the surface treatment of the material layer of the first sensor. Here too, the two material layers may differ and may provide different sensor signals when they are exposed to the same harmful gas.

For example, sulfuric acid (H₂SO₄), hydrochloric acid (HCl), nitric acid (HNO₃), phosphoric acid (H₃PO₄), or sulfonic acid may be used as the acid. The surface treatment may include immersing the material layer of the first sensor in an aqueous solution of the acid. This may be a 1-molar to 10-molar solution of the acid (1 mol/l to 10 mol/l or for example, 3 mol/l to 7 mol/l).

The material layer may be immersed in the acid or the aqueous solution of the acid for about 5 to 90 seconds for the surface treatment, for example, for about 10 to 60 or about 20 to 40 seconds. Immersion is optionally carried out without applying a potential.

To build up the material layer, aniline may be polymerized by applying a dynamic potential. Aniline may be part of an aqueous solution of an acid. For example, polyaniline may be formed in individual, superimposed films on a plate-shaped carrier on which the electrodes are arranged. In an embodiment, the electrodes are interdigital electrodes.

At least the material layer may be functionalized by adding metals, metal alloys or metal oxides (Pd, Ag, Pd/Sn, Zn) to the aqueous solution. For example, it is possible for the material layer of the first sensor to have at least one functionalized film, which the material layer of the second sensor does not have.

For chemical metal deposition, the material layer may be immersed in a dispersion including a solvent and metal nanoparticles.

### BRIEF DESCRIPTION OF THE DRAWINGS

The present disclosure is explained in more detail with reference to embodiments described herein and shown in the accompanying drawings, wherein:

FIG. 1 shows a schematic diagram of an air filter and a fuel cell;

FIG. 2 shows the air filter with a first sensor and a second sensor;

FIG. 3 shows the first sensor with two electrodes and a material layer of polyaniline;

FIG. 4 shows the variation of exposure to the harmful gases NH₃ and NO₂;

FIG. 5 shows the variation of the relative change in resistance over time caused by the exposure to harmful gases in FIG. 4;

FIG. 6 shows the variation of the time derivative of the relative change in resistance caused by the exposure to harmful gases in FIG. 4;

FIG. 7 shows a further variation of exposure to the harmful gas NH₃;

FIG. 8 shows the variation of the relative resistance change caused by the exposure to harmful gas in FIG. 7;

FIG. 9 shows the variation of the time derivative of the relative change in resistance caused by the exposure to the harmful gas in FIG. 7;

FIG. 10 shows the variation of a dynamic resistance ratio based on the exposure to harmful gas in FIG. 7;

FIG. 11 shows a further variation of exposure to the harmful gas NO₂; and

FIG. 12 shows the variation of a dynamic resistance ratio based on the exposure to harmful gas in FIG. 11.

### DETAILED DESCRIPTION

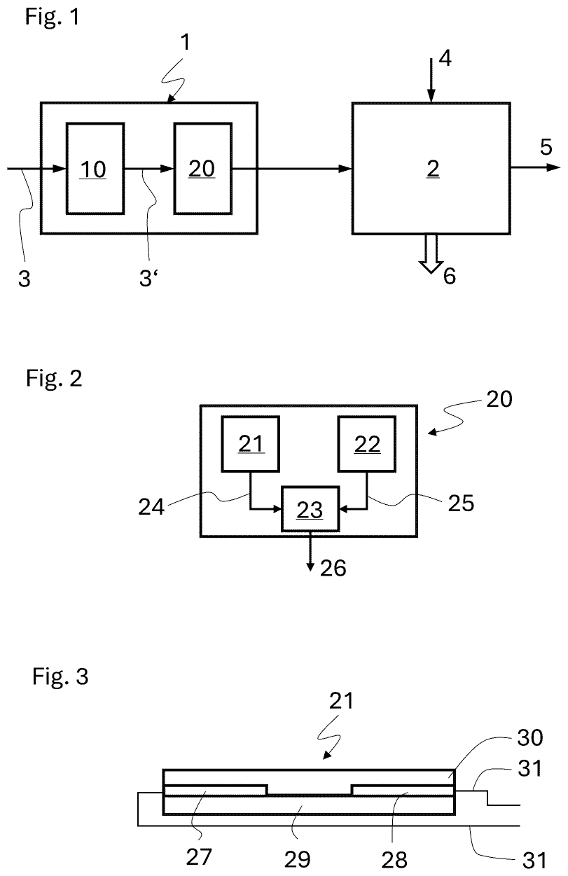

FIG. 1 shows an air filter system 1 and a downstream fuel cell 2. The air filter system 1 comprises an air filter with filter element 10 and a sensor unit 20. An air flow 3 enters the air filter system 1 and passes through the air filter 10. Harmful gases such as nitrogen dioxide (NO₂) and ammonia (NH₃) and/or sulfur dioxide (SO₂) are separated from the air flow 3 by the air filter 10, which comprises adsorbent material. The cleaned air flow 3′ now passes the sensor unit 20, which is used to check the effectiveness of the air filter 10. The sensor unit 20 is designed to detect the harmful gases (e. g., NO₂, NH₃ and/or SO₂) in the ppm range and also in the ppb range. The cleaned cathode air flow 3′ enters the fuel cell 2, where the oxygen in the air flow 3′reacts with an energy carrier such as hydrogen 4 to form water 5, thereby generating electrical energy 6.

FIG. 2 schematically shows the sensor unit 20. The sensor unit 20 comprises a first sensor 21, a second sensor 22 and a processing unit 23. In the processing unit 23, signals 24, 25 from the sensors 21, 22 are processed into an output signal 26. The output signal 26 can, for example, be transmitted to a further processing device (not shown) via a suitable interface. The output signal 26 may then be used, for example, to obtain the information that a harmful gas concentration determined is too high and that the filter element 10 should be replaced as a result.

FIG. 3 schematically shows the structure of the first sensor 21. The structure of the second sensor 22 corresponds to the structure of the first sensor 21, so that only the structure of the first sensor 21 is discussed. The sensor 21 has a first electrode 27 and a second electrode 28, which are arranged on a plate-shaped carrier 29 made of glass. For example, the electrodes are made of gold.

The sensor 21 also has a material layer 30 made of polyaniline, which electrically connects the two electrodes 27, 28. The material layer 30 comprises individual layers or films. Polyaniline is an electrically conductive polymer. When the material layer 30 comes into contact with NH₃ or NO₂, which may be contained in the air flow 3′, the electrical conductivity of the material layer changes, so that the resistance between the first electrode 27 and the second electrode 28 changes. The change in resistance may be measured via electrical lines 31.

The material layer 30 of the first sensor 21 differs from the material layer 30 of the second sensor 22 in that the first sensor 21 with the material layer 30 is immersed in an aqueous solution of sulfuric acid for a certain period of time. This immersion in the aqueous solution changes the properties, for example, reactive properties, of the material layer 30 of the first sensor 21 compared to the untreated material layer of the second sensor 22.

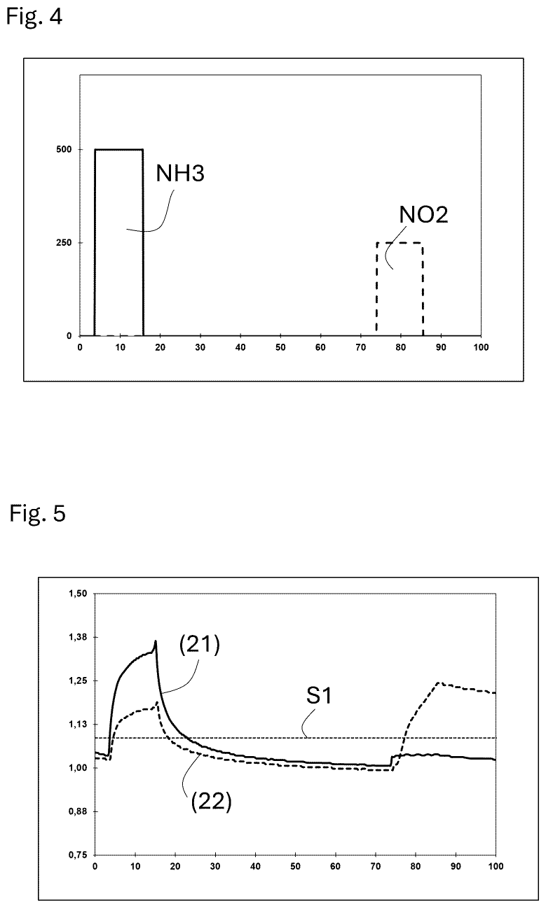

For test purposes, the sensor unit 20 is exposed to the harmful gas NH₃ and, with a time delay, to the harmful gas NO₂. The diagram in FIG. 4 shows the selected concentration in ppb, the exposure duration (10 min), and the time interval (60 min) between the first exposure to NH₃ and the second exposure to NO₂. The air flow 3′ is guided to the two sensors 21, 22 with an NH₃ concentration of 500 ppb and, after a break of 60 minutes, with an NO₂ concentration of 250 ppb.

The solid line in the diagram in FIG. 5 corresponds to the reaction of the first sensor 21. The dashed line corresponds to the sensor reaction of the second sensor 22. The time in minutes is plotted on the x-axis and the relative change in resistance R/R0 on the y-axis, where R0 corresponds to the initial resistance of the material layer of the two sensors 21 and 22 before exposure to the harmful gas. It is seen that the first sensor 21, which was post-treated with H₂SO₄, shows a stronger reaction when exposed to NH₃ than the second sensor 22, which was not post-treated with H₂SO₄. After exposure to NH₃, the sensor signal of the two sensors 21 and 22 returns to at least substantially the same level.

When exposed to NO₂, the sensor 21 post-treated with H₂SO₄ shows practically no reaction, while the untreated sensor 22 shows a clear reaction (the relative change in resistance R/R0 increases to approx. 1.25). After the increase to the value 1.25, however, the relative change in resistance remains at this level and does not return to anywhere near the original value of 1.0. A threshold value S1 for the relative change in resistance R/R0 is shown as an example in the diagram in FIG. 5. The threshold value S1 here is approx. 1.1. While this threshold value is reached by both sensors when NH₃ is applied, only the sensor signal of the untreated sensor 22 is above this threshold value S1 when NO₂ is applied.

Based on the comparison with the threshold value S1 and the sensor signal of the two sensors 21, 22, a statement could be made in real operation as to whether the sensor unit has been exposed to NH₃ or NO₂. If both the first sensor 21 and the second sensor 22 show a sensor response above the threshold value S1, it may be concluded that the harmful gas is NH₃. The processing unit may thus generate a corresponding output signal.

If, on the other hand, only the second sensor 22 shows a reaction above the threshold value S1, it may be concluded that the harmful gas is NO₂. The processing unit would now generate a correspondingly different output signal.

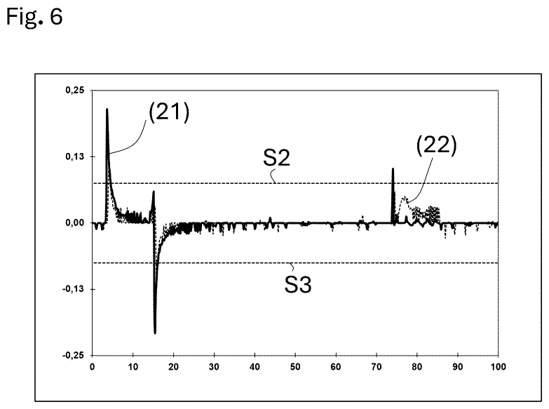

FIG. 6 shows the course of the time derivative of the relative resistance change Δ(R/R0)/Δt over time. As in FIG. 5, the corresponding sensor response as a result of the exposures shown in FIG. 4 is also shown here.

FIG. 6 clearly shows that a bipolar pulse may be assigned to both sensors when exposed to NH₃. Both the first sensor (post-treated with H₂SO₄) and the second sensor 22 show a pulse peak at the beginning of the NH₃ exposure. When this exposure ends, a negative pulse peak may be recognized, which corresponds approximately to the absolute amount of the initial positive pulse peak.

Exposure to NO₂, on the other hand, only results in a unipolar pulse. A negative pulse peak at the end of the NO₂ exposure is not recognizable.

In the diagram in FIG. 6, a second threshold value S2 is shown as an example for the positive pulse peaks and a third negative threshold value S3 for the negative pulse peaks. The amount of the threshold values may be the same or different. If both sensors 21, 22 now each detect a bipolar pulse, whereby the respective positive pulse peak is above the second threshold value S2 and the respective negative pulse peak is below the third threshold value S3, this is an indication of the harmful gas NH₃. By checking the corresponding conditions, the processing unit may generate the output signal that is derived from this.

If the sensor signal from the two sensors 21, 22 does not have a negative pulse peak (condition relating to threshold value S3 not fulfilled) and at the same time the condition relating to the relative resistance change R/R0 is met, i.e. the sensor signal from the second sensor 22 is greater than S1 and the sensor signal from the first sensor 21 is less than S1, the processing unit determines an output signal that indicates the presence of NO₂.

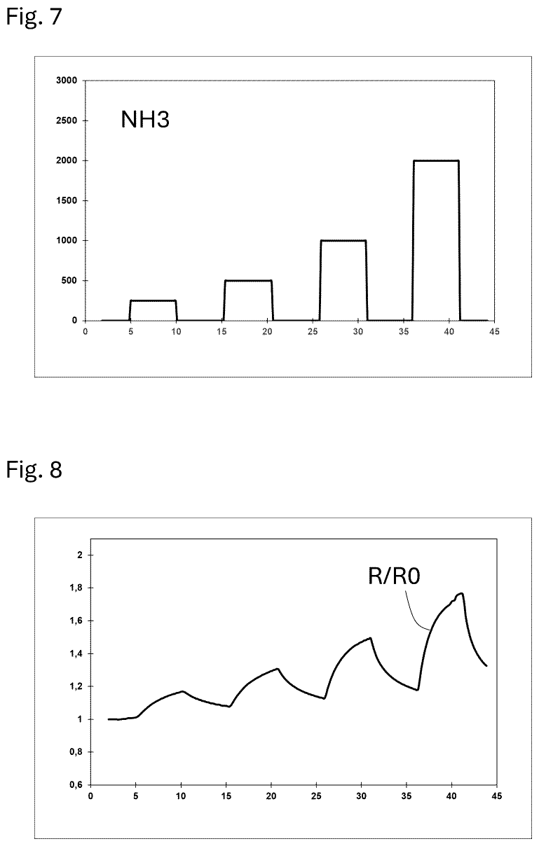

FIG. 7 shows the variation of exposure of a polyaniline sensor to NH₃. Starting at a concentration of 250 ppb, the gas concentration is gradually increased to 500, 1000 and 2000 ppb. Between the individual stages of harmful gas exposure, each lasting 5 minutes, there are breaks of 5 minutes.

The resistance of the polyaniline sensor changes as a result of exposure to harmful gases, as shown in FIG. 7.

FIG. 8 shows the relative resistance change R/RO over time.

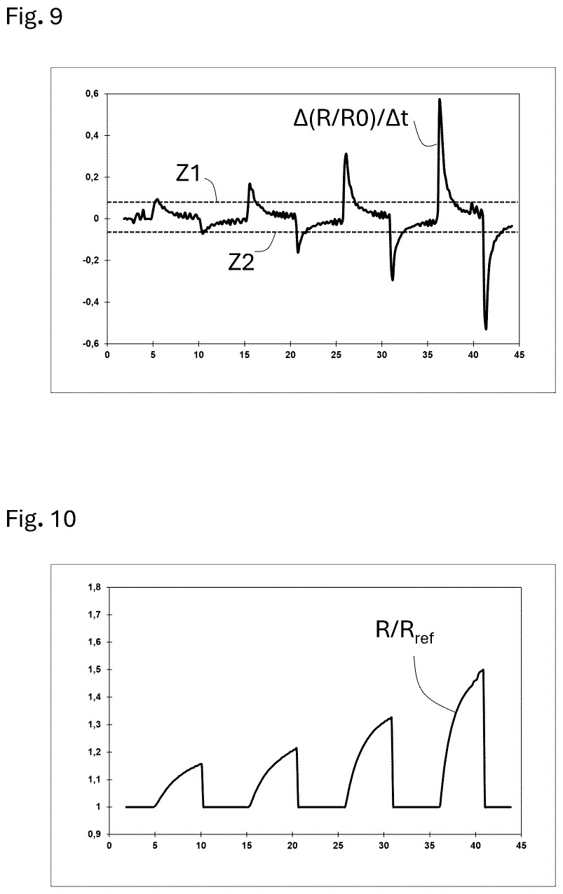

FIG. 9 shows the time derivative of the relative change in resistance in FIG. 8 (Δ(R/R0)/Δt). Due to the first stage of exposure to harmful gases, the time curve of Δ(R/R0)/Δt exceeds a start threshold value Z1 at approximately 5 to 6 minutes (see upper dashed line in FIG. 9). When the first stage of exposure ends after approximately 5 minutes, the time derivative of the relative change in resistance Δ(R/R0)/Δt drops to values below zero and falls below an end threshold value Z2 (see lower dashed line in FIG. 9). The start threshold value Z1 is between 0 and 0.2 s−1 , while the final threshold value is less than zero. In the second stage of harmful gas exposure (approx. between 15 and 20 minutes), the start threshold value Z1 is first exceeded and then the end threshold value Z2 is again undershot. Exceeding the start threshold value Z1 and falling below the end threshold value Z2 is repeated in the third and fourth stages of harmful gas exposure.

FIG. 10 shows the variation of a dynamic resistance ratio R/R_{ref}. The numerator of the dynamic resistance ratio is the resistance measured by the sensor. The denominator corresponds to a dynamic baseline resistance R_{ref}, which is determined by Δ(R/R0)/Δt and the threshold values Z1 and Z2. The dynamic baseline resistance R_{ref} may be set and reset based on the time derivative Δ(R/R0)/Δt and the threshold values Z1 and Z2.

As long as the start threshold value Z1 has not been exceeded starting from time t=0, the dynamic baseline resistance R_{ref} corresponds to the measured resistance R(t). Accordingly, until the point in time when the time curve of Δ(R/R0)/Δt exceeds the start threshold value Z1 for the first time, the dynamic resistance ratio R/R_{ref} equals 1. After exceeding the start threshold value Z1, the dynamic resistance ratio increases to approx. 1.15. In this phase from approx. t=5 min to approx. t=10 min, the dynamic baseline resistance R_{ref} is constant and corresponds to the resistance determined by the sensor at the intersection of the start threshold value Z1 with the time curve Δ(R/R0)/Δt. At the point in time at approx. t=10 min, when the value falls below the end threshold value Z2, the dynamic baseline resistance is again equated with the measured resistance R(t), whereby the ratio R/R_{ref} returns to the value 1. Due to the repeated exceeding of Z1 and the subsequent falling below Z2 (see FIG. 9), the dynamic resistance ratio R/R_{ref} (see FIG. 10) is reset to the value 1 after each stage of harmful gas exposure. The dynamic resistance ratio R/R_{ref} may be correlated with the values of the harmful gas exposure (see FIG. 7), so that based on this correlation and the temporal course of the dynamic resistance ratio R/R_{ref}, the sensor unit may display specific values for the harmful gas concentration.

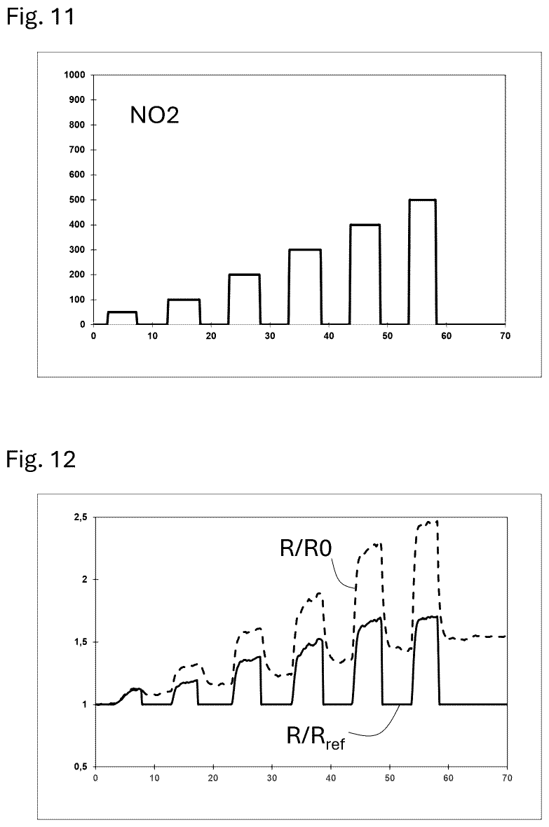

FIG. 11 shows another time curve of harmful gas exposure with NO₂. A sensor with a metal oxide (Mox) layer is exposed to six stages with different concentrations ranging from 50 to 500 ppb. FIG. 12 shows the time curve of the relative resistance change R/RO (dashed line) and the time curve of the dynamic resistance ratio R/R_{ref}, whereby the value R/R_{ref} was calculated in analogy to the embodiment shown in FIGS. 7 to 10. The use of R/R_{ref} for the output signal allows sensor drift to be compensated. It has also been shown that the use of R/R_{ref} may effectively compensate for possible influences of changes in relative humidity and temperature.

Determining the time derivative of the relative change in resistance enables a better and more accurate statement to be made about the harmful gases detected. Due to the usually slowly changing temperature and humidity conditions, mathematical compensation of the sensor signals is not necessary when using the time derivative Δ(R/R0)/Δt. This results in a simple sensor unit with high measurement accuracy. Due to the surface treatment of the material layer of only one sensor with an otherwise identical structure of the material layer of both sensors, the processing unit of the sensor unit differentiates between NH₃ and NO₂.

According to various embodiments, the sensor unit may comprise more than two sensors, for example three, four or a larger array of sensors, each having material layers with different compositions and/or surface treatments. By providing multiple sensors with deliberately varied sensitivities and selectivities to NH₃, NO₂ and other harmful gases, the processing unit may evaluate a richer pattern of resistance changes and time derivatives. This enables improved gas discrimination, more robust detection under varying environmental conditions, and enhanced redundancy. If one sensor drifts or fails, the remaining sensors may still provide reliable information about harmful gases.

### REFERENCE CHARACTers

1 air filter system
2 fuel cell
3 air flow (3′ clean air flow)
4 hydrogen
5 water
6 energy
10 air filter
20 sensor unit
21 first sensor
22 second sensor
23 processing unit
24 sensor signal
25 sensor signal
26 output signal
27 first electrode
28 second electrode
29 carrier
30 material layer
31 line

That which is claimed is:

1. A sensor unit for use in an air filter system, the sensor unit operable to
separate at least one harmful gas from an air flow, the sensor unit comprising:
a first sensor comprising
two electrodes, and
a material layer connecting the two electrodes, wherein an electrical resistance of the material layer changes when the material layer comes into contact with the at least one harmful gas; and
a processing unit configured to determine a time derivative of a change in an electrical resistance of the material layer of the first sensor, and further configured to generate an output signal based on a function of the time derivative of the first sensor determined by the processing unit.

2. The sensor unit according to claim 1, wherein the material layer is formed from polyaniline or metal oxide.

3. The sensor unit according to claim 1, further comprising:
a second sensor comprising
two further electrodes, and
a further material layer connecting the two further electrodes,
wherein the material layer of the first sensor is different from the further material layer of the second sensor, and
wherein the processing unit is further configured to determine a time derivative of a change in an electrical resistance of the further material layer of the second sensor, and is further configured to generate the output signal further based on a function of the time derivative of the second sensor determined by the processing unit.

4. The sensor unit according to claim 3, wherein the processing unit is further configured to identify the at least one harmful gas based on comparing the respective electrical resistances of the material layer of the first sensor and the further material layer of the second sensor.

5. The sensor unit according to claim 1, wherein the sensor unit is configured to generate the output signal based on whether the change in electrical resistance of the material layer of the first sensor exceeds a threshold value S1.

6. The sensor unit according to claim 1, wherein the sensor unit is configured to generate the output signal based on whether the time derivative of the change in electrical resistance of the material layer of the first sensor exceeds a threshold value S2.

7. The sensor unit according to claim 1, wherein the sensor unit is configured to generate the output signal based on whether the time derivative of the change in electrical resistance of the material layer of the first sensor falls below a negative threshold value S3.

8. The sensor unit according to claim 1, wherein the output signal is based on a dynamic resistance ratio R/R_{ref}, wherein R is a measured electrical resistance of the material layer and R_{ref} is a dynamic baseline resistance of the material layer that depends on the time derivative of the change in electrical resistance of the material layer of the first sensor determined by the processing unit.

9. The sensor unit according to claim 8, wherein the dynamic baseline resistance R_{ref} is defined as follows:
R_{ref} =measured electrical resistance R(t), as long as a time derivative of the relative change in resistance Δ(R/R0)/Δt with R0 as the initial resistance, is smaller than a start threshold value Z1;
R_{ref} =measured electrical resistance R(Z1) at a point at which the time derivative of the relative change in resistance Δ(R/R0)/Δt exceeds the start threshold value Z1 and as long as the time derivative of the relative change in resistance Δ(R/R0)/Δt does not fall below an end threshold value Z2; and
R_{ref} =measured resistance R(t) as soon as the time derivative of the relative change in resistance Δ(R/R0)/Δt falls below the end threshold value Z2.

10. The sensor unit according to claim 8, wherein the output signal is indicative of a concentration of the at least one harmful gas, and wherein the concentration is determined based on a calibration with the dynamic resistance ratio R/R_{ref}.

11. A method for producing a sensor unit according to claim 1, wherein the material layer of the first sensor is subjected to a surface treatment with an acid.

12. The method according to claim 11, wherein the acid comprises at least one of sulfuric acid (H₂SO₄), hydrochloric acid (HCl), nitric acid (HNO₃), phosphoric acid (H₃PO₄) and sulfonic acid.

13. The method according to claim 11, wherein the surface treatment comprises immersing the material layer of the first sensor in a 1-molar to 10-molar aqueous solution of the acid.

14. The method according to claim 11, wherein aniline in an aqueous solution of the acid is polymerized by applying a dynamic potential to build up the material layer.

15. The method according to claim 14, wherein the material layer is functionalized by mixing at least one of metals, metal alloys and metal oxides into the aqueous solution.

16. The method according to claim 14, wherein for chemical metal deposition, the material layer is immersed in a dispersion comprising a solvent and metal nanoparticles.
